(19) United States
(12) Patent Application Publication
(10) Pub. No.: US 2025/0166297 A1
(43) Pub. Date: May 22, 2025

(54) IMAGE PROCESSING APPARATUS, IMAGE PROCESSING METHOD, AND STORAGE MEDIUM

(71) Applicant: OLYMPUS MEDICAL SYSTEMS CORP., Tokyo (JP)

(72) Inventor: Yuma MASUDA, Tokyo (JP)

(73) Assignee: OLYMPUS MEDICAL SYSTEMS CORP., Tokyo (JP)

(21) Appl. No.: 18/952,452

(22) Filed: Nov. 19, 2024

Related U.S. Application Data
(60) Provisional application No. 63/602,000, filed on Nov. 22, 2023.

(51) Int. Cl. G06T 17/00; G06T 7/00; G06V 10/25

(52) U.S. Cl. CPC G06T 17/00; G06T 7/0012; G06V 10/25; G06T 2207/10068

(57) ABSTRACT

An image processing apparatus includes one or more processors. The one or more processors comprise hardware. The one or more processors are configured to acquire a plurality of images of an object captured by an endoscope, detect a first region of the object included in the plurality of images, generate a partial three-dimensional model of the object based on a one or more images of the plurality of images, the partial three-dimensional model representing the first region, and detect, based on the partial three-dimensional model, an unobserved region of the object that is not captured by the endoscope.

### CROSS-REFERENCE TO RELATED APPLICATIONS

This application claims the benefit of U.S. Provisional Application No. 63/602,000, filed on Nov. 22, 2023, which is hereby incorporated by reference herein in its entirety.

### TECHNICAL FIELD

The present disclosure relates to an image processing apparatus, an image processing method, and a storage medium.

### BACKGROUND ART

Conventionally, a known technique in the related art reconstructs a three-dimensional model of an object from an image group obtained during an endoscopic examination (for example, see PTL 1). In PTL 1, an unobserved region in a three-dimensional model is detected, and the unobserved region is displayed in a visible manner. The unobserved region is a region that has not been observed by the endoscope. This technique is useful for detecting an overlooked region in an endoscopic examination.

### CITATION LIST

### Patent Literature

PTL 1: Publication of Japanese Patent No. 6242543

### SUMMARY

An aspect of the disclosure is an image processing apparatus comprising one or more processors comprising hardware, the one or more processors are configured to: acquire a plurality of images of an object captured by an endoscope; detect a first region of the object included in the plurality of images; generate a partial three-dimensional model of the object based on one or more images of the plurality of images, the partial three-dimensional model representing the first region; and detect, based on the partial three-dimensional model, an unobserved region of the object that is not captured by the endoscope.

Another aspect of the disclosure is an image processing method comprising: acquiring a plurality of images of an object captured by an endoscope; detecting a first region of the object included in the plurality of images; generating a partial three-dimensional model of the object based on one or more images of the plurality of images, the partial three-dimensional model representing the first region of interest; and detecting, based on the partial three-dimensional model, an unobserved region of the object that is not captured by the endoscope.

Another aspect of the disclosure is a non-transitory computer-readable storage medium storing an image processing program. The image processing program causes a computer to execute: acquiring a plurality of images of an object captured by an endoscope; detecting a first region of interest included in the plurality of images; generating a partial three-dimensional model of the object based on one or more images of the plurality of images, the partial three-dimensional model representing the first region; and detecting, based on the partial three-dimensional model, an unobserved region of the object that is not captured by the endoscope.

### BRIEF DESCRIPTION OF DRAWINGS

FIG. 1 is a block diagram showing the overall configuration of an endoscope system according to a first embodiment.

FIG. 2 is a functional block diagram of a processor of an image processing apparatus according to the first embodiment.

FIG. 3 illustrates a procedure for a colonoscopy.

FIG. 4 illustrates a whole 3D model generated in a colonoscopy.

FIG. 5A illustrates an example indication indicating an unobserved region in an image.

FIG. 5B illustrates another example indication indicating an unobserved region in an image.

FIG. 6A illustrates an example indication indicating that an unobserved region has been detected.

FIG. 6B illustrates another example indication indicating that an unobserved region has been detected.

FIG. 7 is a flowchart of an image processing method according to the first embodiment.

FIG. 8A illustrates a method for detecting a fold from a 3D model.

FIG. 8B illustrates a method for detecting a fold from a 3D model.

FIG. 8C illustrates an image corresponding to a slice of FIG. 8B.

FIG. 9 illustrates a distribution of point clouds representing folds in a plurality of images.

FIG. 10 is a functional block diagram of a processor of an image processing apparatus according to a second embodiment.

FIG. 11 is a flowchart of an image processing method according to the second embodiment.

FIG. 12 is a functional block diagram of a processor of an image processing apparatus according to a third embodiment.

FIG. 13 is a flowchart of an image processing method according to the third embodiment.

### DESCRIPTION OF EMBODIMENTS

Images input from an endoscope to an image processing apparatus include images of various scenes. Conventionally, the input images are used to generate a three-dimensional model regardless of the scenes. For example, it is difficult to generate an accurate three-dimensional model of an object with images of a scene in water, a scene in which bubbles are attached to a lens, and the like, and such images may cause erroneous detection of an unobserved region.

### First Embodiment

An image processing apparatus, an image processing method, an image processing program, and a storage medium according to a first embodiment will be described with reference to the drawings.

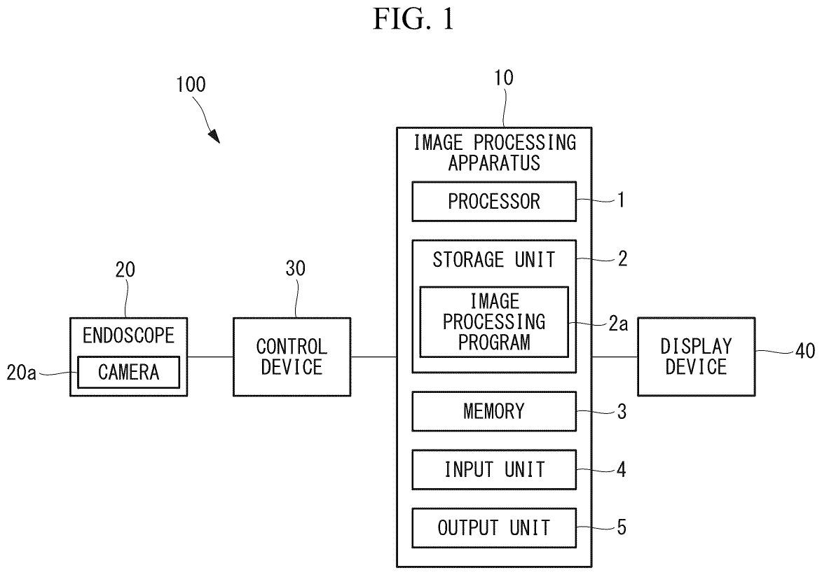

As shown in FIG. 1, an image processing apparatus 10 according to this embodiment is applied to an endoscope system 100.

The endoscope system 100 includes the image processing apparatus 10, an endoscope 20, a control device 30, and a display device 40.

The endoscope 20 is, for example, a flexible endoscope for digestive organs such as the colon. The endoscope 20 has a two-dimensional camera 20a at the distal end thereof and captures a two-dimensional image of an object with the camera 20a. The control device 30 is connected to the endoscope 20 and controls illumination light and the like to be supplied to the endoscope 20. The image captured by the endoscope 20 is input to the display device 40 through the control device 30 and the image processing apparatus 10 and is displayed on the display device 40. The display device 40 is a known display such as a liquid crystal display.

The image processing apparatus 10 includes a processor 1 such as a central processing unit, a storage unit 2, a memory 3, an input unit 4, and an output unit 5. The image processing apparatus 10 is composed of, for example, a personal computer.

The storage unit 2 is a non-transitory computer-readable storage medium, and examples thereof include a known magnetic disk, optical disk, and flash memory. The storage unit 2 stores an image processing program 2a for causing the processor 1 to perform the image processing method described below.

The memory 3 is a volatile storage device, such as a random access memory (RAM), and is used as a work area for the processor 1.

The input unit 4 includes a known input interface and is connected to the control device 30. The output unit 5 includes a known output interface and is connected to the display device 40. An image is input to the image processing apparatus 10 through the input unit 4 and is output to the display device 40 through the output unit 5.

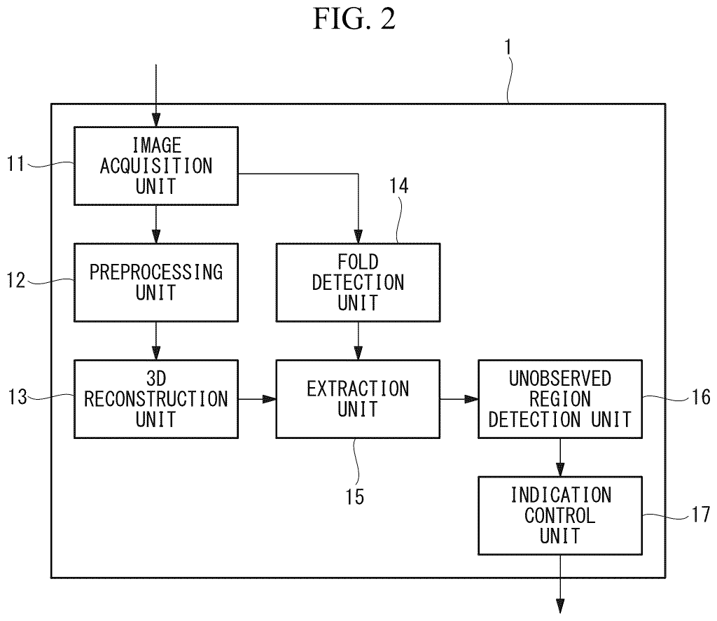

As shown in FIG. 2, the processor 1 includes, as functional units, an image acquisition unit 11, a preprocessing unit 12, a three-dimensional (3D) reconstruction unit 13, a fold detection unit 14, an extraction unit 15, an unobserved region detection unit 16, and an indication control unit 17.

While the endoscope 20 is operating, time-series continuous images constituting a moving image are input from the endoscope 20 to the image processing apparatus 10.

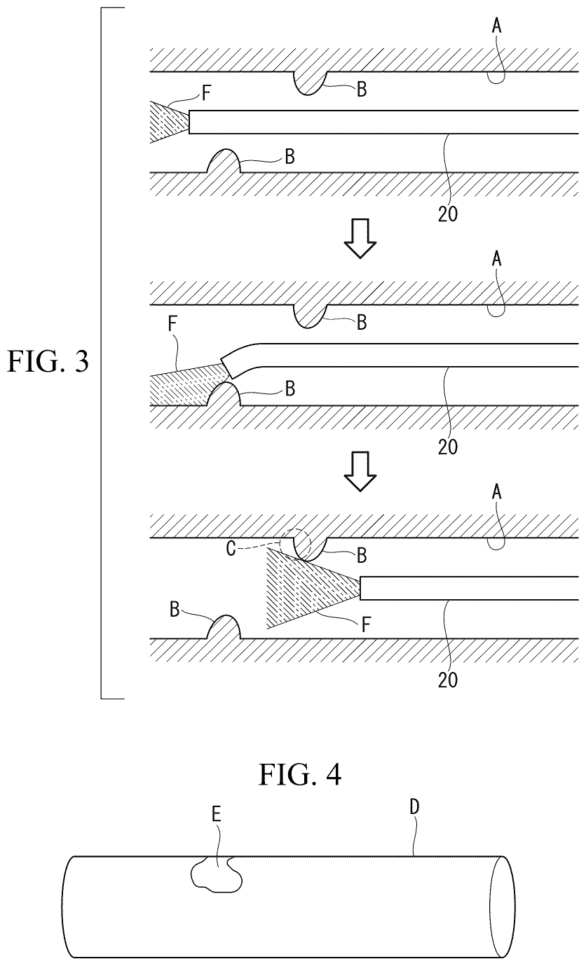

FIG. 3 illustrates a colonoscopy. In a typical colonoscopy, a user such as a doctor inserts the endoscope 20 from the anus to the cecum, and then inspects each site of the colon on the basis of the image while removing the endoscope 20 from the cecum to the anus. Hence, during the examination, continuous images of an object A, such as the colon, captured at different positions are input to the image processing apparatus 10.

The image acquisition unit 11 sequentially acquires images input to the image processing apparatus 10 to obtain a plurality of images. The plurality of images are images with which a 3D model of the object A continuous in the movement direction of the field of view of the endoscope 20 can be generated. The image acquisition unit 11 may acquire all the images input to the image processing apparatus 10. In that case, the plurality of images are continuous images constituting a moving image. Alternatively, the image acquisition unit 11 may selectively acquire images input to the image processing apparatus 10. In that case, the plurality of images are intermittent images discretely arranged along the movement direction of the endoscope 20.

The preprocessing unit 12 performs preprocessing such as distortion correction on the images acquired by the image acquisition unit 11. The preprocessed images are at least temporarily stored in the storage unit 2, the memory 3, or another storage device. Thus, an image group including a plurality of preprocessed images is obtained.

As shown in FIG. 4, the 3D reconstruction unit 13 generates a three-dimensional (3D) model D of the object A from the image group. Specifically, the 3D reconstruction unit 13 estimates a three-dimensional shape of the object A from the image group and reconstructs the three-dimensional shape by using a known three-dimensional reconstruction technique, such as SLAM. The generated 3D model D is a whole 3D model of the object A included in the image group and has a tubular shape when the object A is a lumen such as the colon.

The fold detection unit 14 detects a predetermined region of interest of the object included in the image group. Specifically, the predetermined region of interest is a fold B protruding from the inner wall of the colon A (see FIG. 3). The fold detection unit 14 detects a fold B from each of the plurality of images G (see FIGS. 5A and 5B). The predetermined region of interest is the region in which detection of the presence/absence of an unobserved region C is performed, as described below. In the colonoscopy, the reverse side of a fold B is often a blind spot for the endoscope 20. Thus, it is important to prevent a doctor from overlooking the reverse side of the fold B. Accordingly, a fold B is set as the region of interest.

In one example, the fold detection unit 14 may recognize a fold B in an image G by using a learning model. The learning model is generated by deep learning using images in which the regions of folds are annotated, and is stored in the storage unit 2 in advance.

In another example, the fold detection unit 14 may detect an edge in the image G to detect a fold B on the basis of the edge.

The extraction unit 15 extracts, from the whole 3D model D generated by the 3D reconstruction unit 13, a portion corresponding to the region of the fold B detected by the fold detection unit 14 to generate a partial 3D model. The partial 3D model generated in this way is composed of the fold B portion and is based on a part of the image group.

The unobserved region detection unit 16 detects an unobserved region C on the basis of the partial 3D model generated by the extraction unit 15. The unobserved region C is a region that has never been captured by the endoscope 20. In the examples of FIGS. 3 and 4, an unobserved region C is present behind a fold B. The unobserved region C forms a missing part E, which is a hole where the shape of the object A is not restored, in the 3D model D. The unobserved region detection unit 16 may detect a missing part E in the partial 3D model to regard it as an unobserved region.

The unobserved region detection unit 16 may detect an unobserved region on the basis of the positional relationship between a fold B and a position the field of view F of the endoscope 20 in the whole or partial 3D model.

Specifically, in the three-dimensional reconstruction process, the position and the orientation of the camera 20a in the whole 3D model D are calculated. The unobserved region detection unit 16 calculates the position of the field of view F in the whole or partial 3D model from the position and the orientation of the camera 20a, and determines whether or not the reverse side of the fold B is included in the field of view F on the basis of the positional relationship between the field of view F and the fold B in the whole or partial 3D model. Then, the unobserved region detection unit 16 detects the reverse side of the fold B as an unobserved region when the reverse side of the fold B is not included in the field of view F, and determines that the reverse side of the fold B has been observed when the reverse side of the fold B is included in the field of view F.

When the unobserved region detection unit 16 has detected an unobserved region, the indication control unit 17 generates an indication H and outputs the indication H together with the image G to the display device 40 through the output unit 5 to cause the display device 40 to display the indication H in real time. The indication H indicates that an unobserved region has been detected.

As shown in FIGS. 5A and 5B, when the detected unobserved region is included in the image G, the indication control unit 17 may generate an indication H indicating the position of the unobserved region in the image G to be superimposed on the image G. The indication H may be an arrow pointing to the unobserved region (see FIG. 5A), or may be a marker superimposed at a position corresponding to the unobserved region (see FIG. 5B). The arrow H in FIG. 5A points to the fold B behind which an unobserved region has been detected, and the marker H in FIG. 5B is superimposed on the front side of the fold B behind which an unobserved region has been detected.

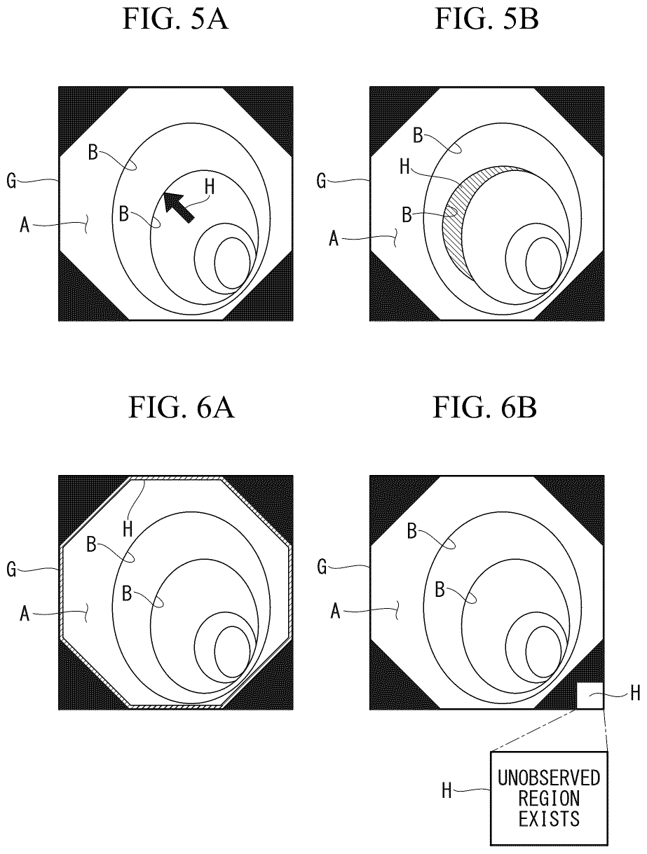

As shown in FIGS. 6A and 6B, when the detected unobserved region does not appear in the image G, the indication control unit 17 may indicate, as the indication H, an alert notifying the presence of the unobserved region. The alert H may be indicated outside the image G. For example, the alert H may be a frame surrounding the image G (see FIG. 6A) or may be a text (see FIG. 6B).

The indication H may be a guide to the unobserved region, which may be, for example, an indication of the distance from the distal end of the endoscope 20 to the unobserved region or operation process to reach the unobserved region.

Next, an image processing method performed by the image processing apparatus 10 will be described.

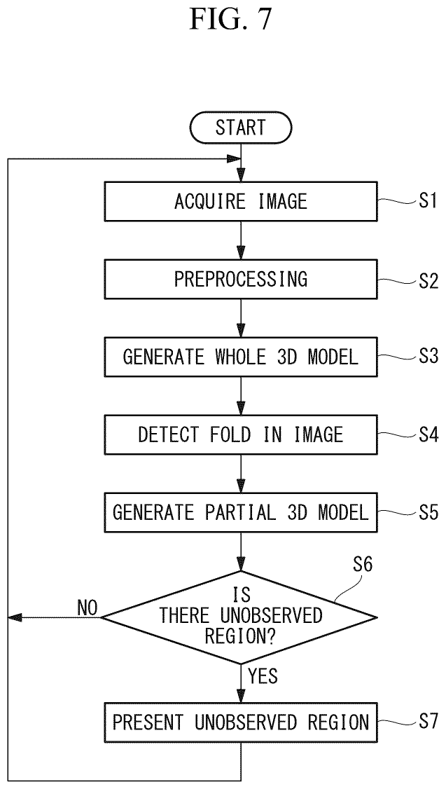

As shown in FIG. 7, the image processing method according to this embodiment includes: step S1 of acquiring images of the object A captured by the endoscope 20; step S2 of preprocessing the images; step S3 of generating a whole 3D model D of the object A; step S4 of detecting a predetermined region of interest of the object A from the images; step S5 of generating a partial three-dimensional model of the object A; step S6 of detecting an unobserved region on the basis of the partial three-dimensional model; and step S7 of presenting information of the detected unobserved region to a user.

For example, in a colonoscopy, images captured by the endoscope 20 while the endoscope is removed from the cecum toward the anus are input to the image processing apparatus 10.

The image acquisition unit 11 sequentially acquires the images input to the image processing apparatus 10 (step S1), and then the preprocessing unit 12 sequentially preprocesses the images acquired by the image acquisition unit 11 (step S2). As a result, an image group including a plurality of preprocessed images is obtained. Next, the 3D reconstruction unit 13 generates a whole 3D model D of the object A from the image group (step S3).

As a result of steps S2 and S3 being performed each time a new image is acquired, a whole 3D model D of the object A that has been captured by the endoscope 20 by that time is generated in real time.

In parallel with steps S2 and S3, the fold detection unit 14 detects a fold B in the images acquired by the image acquisition unit 11 (step S4). As a result of step S4 being performed each time a new image is acquired by the image acquisition unit 11, the region of the fold B in the plurality of images that are used for generating the whole 3D model D is detected.

Next, the extraction unit 15 extracts, from the whole 3D model D, a portion corresponding to the region of the fold B detected from the plurality of images to obtain a partial 3D model of the object A (step S5).

Next, the unobserved region detection unit 16 detects the presence/absence of an unobserved region on the basis of the partial 3D model (step S6).

When no unobserved region has been detected (NO in step S6), the process repeats steps S1 to S6 without performing step S7.

When an unobserved region has been detected (YES in step S6), the indication control unit 17 presents the information of the detected unobserved region to the user (step S7). Specifically, the indication control unit 17 generates an indication H, such as an arrow or a marker, indicating that an unobserved region has been detected, and outputs the indication H to the display device 40 together with the image G. The user can recognize the presence of the unobserved region C on the basis of the indication H displayed on the display device 40 in real time.

The moving image input to the image processing apparatus 10 during an endoscopic examination may include an image of a scene that is not suitable for detection of an unobserved region. For example, an image of a scene in which the object is unclear or partially invisible, such as a scene in water, a scene in which bubbles are attached to a lens, and a scene including a residue, is not suitable because it is difficult to generate an accurate 3D model, potentially leading to erroneous detection of an unobserved region.

According to this embodiment, a portion corresponding to the region of interest is extracted from the whole 3D model D on the basis of the region of interest detected from the image G. Hence, even when the image group acquired by the processor 1 includes an image of an unsuitable scene, a partial 3D model of the region of interest, excluding an inaccurate portion resulting from an image of an unsuitable scene, is generated. By using this partial 3D model for detection of an unobserved region, it is possible to prevent erroneous detection of an unobserved region and prevent erroneous information of an unobserved region from being presented to the user. It is also possible to prevent information of an unobserved region, such as information of an unobserved region other than the region of interest, from being excessively presented to the user.

In a typical colonoscopy, an unobserved region C is likely to occur behind a fold B, which is a blind spot. Because the region of interest is the region of the fold B, it is possible to effectively prevent the user from overlooking the reverse side of the fold B in the colonoscopy.

In this embodiment, the fold detection unit 14 detects a fold B from a two-dimensional image G. Instead of this or in addition to this, a fold B may be detected from a whole 3D model D.

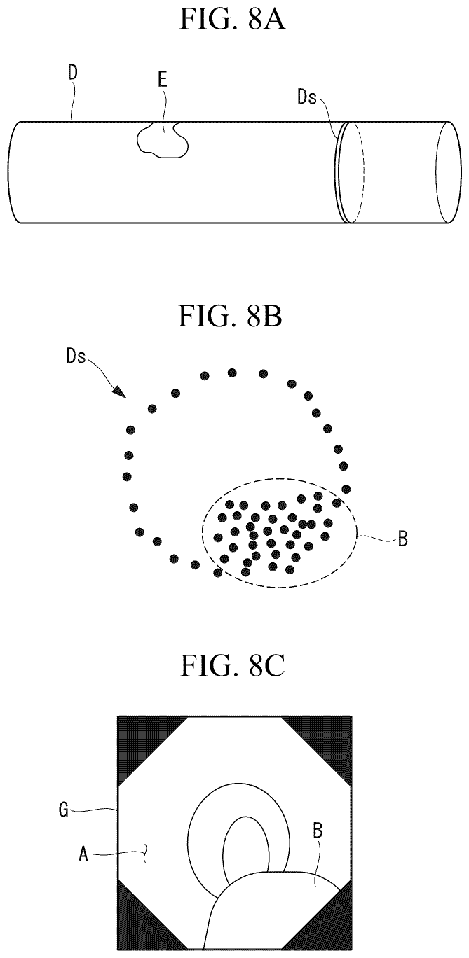

For example, as shown in FIGS. 8A and 8B, the fold detection unit 14 slices the whole 3D model D in a direction orthogonal to the longitudinal direction thereof, and generates a slice Ds of the 3D model D. The 3D model D includes a point cloud representing the surface of the object A. In the slice Ds, the density of the point cloud at the portion corresponding to the fold B is higher than that of the other portion. FIG. 8C shows an image G corresponding to the slice Ds in FIG. 8B. The fold detection unit 14 detects a portion where the density of the point cloud is high, which is regarded as a fold B. In this way, a 3D model D generated from an image group may be used to detect a fold B included in the image group.

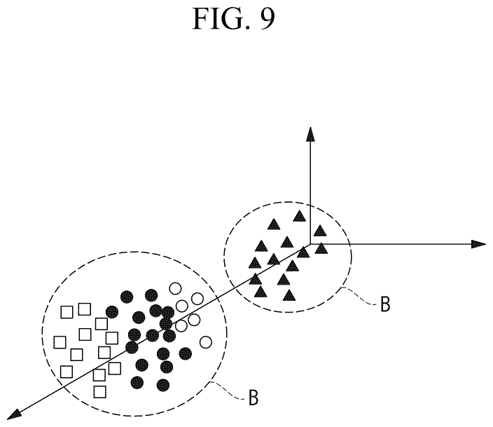

When detection of a fold B from an image G and detection of a fold B from a 3D model D are combined, as shown in FIG. 9, the fold detection unit 14 may determine a point cloud corresponding to a fold B in the 3D model D on the basis of the fold B detected from a plurality of images G.

Specifically, the fold detection unit 14 extracts, from the 3D model D, a point cloud corresponding to the region of a fold B detected from each of the plurality of images G and recognizes the point clouds corresponding to the region of the same fold B in the plurality of images G as a single fold. FIG. 9 shows a distribution of the point clouds in a three-dimensional space. The square, black circle, white circle and triangle point clouds indicate the point clouds corresponding to folds B in different images G.

The 3D model D may have a region in which a high-density point cloud exists, in addition to a fold B. A fold B can be detected more accurately on the basis of both the image G and the 3D model D.

### Second Embodiment

Next, an image processing apparatus, an image processing method, an image processing program, and a storage medium according to a second embodiment will be described.

This embodiment differs from the first embodiment in the method of generating a partial 3D model. In this embodiment, the configurations different from those in the first embodiment will be described, and the configurations common to those in the first embodiment will be denoted by the same reference signs, and the description thereof will be omitted.

As in the first embodiment, the image processing apparatus 10 according to this embodiment is applied to the endoscope system 100 including the endoscope 20, the control device 30, and the display device 40. The image processing apparatus 10 includes a processor 101, the storage unit 2, the memory 3, the input unit 4, and the output unit 5.

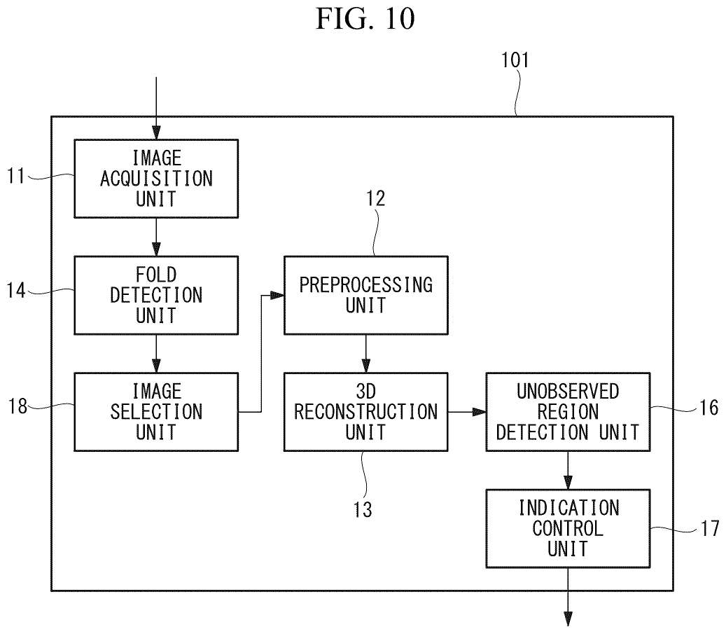

As shown in FIG. 10, the processor 101 includes, as functional units, an image selection unit 18 in addition to the image acquisition unit 11, the preprocessing unit 12, the 3D reconstruction unit 13, the fold detection unit 14, the unobserved region detection unit 16, and the indication control unit 17.

The image acquisition unit 11 sequentially acquires images input to the image processing apparatus 10 to obtain a plurality of images.

The fold detection unit 14 detects a fold B in each of the images acquired by the image acquisition unit 11.

The image selection unit 18 selects only the images in which a fold B has been detected by the fold detection unit 14, so that the selected images are used for 3D model generation.

The 3D reconstruction unit 13 generates a 3D model of an object A from the plurality of images selected by the image selection unit 18. The thus-generated 3D model is a partial 3D model representing the fold B and the peripheral portion thereof and is based on some of the plurality of images.

The unobserved region detection unit 16 detects an unobserved region on the basis of the partial 3D model generated by the 3D reconstruction unit 13.

Next, an image processing method performed by the image processing apparatus 10 will be described.

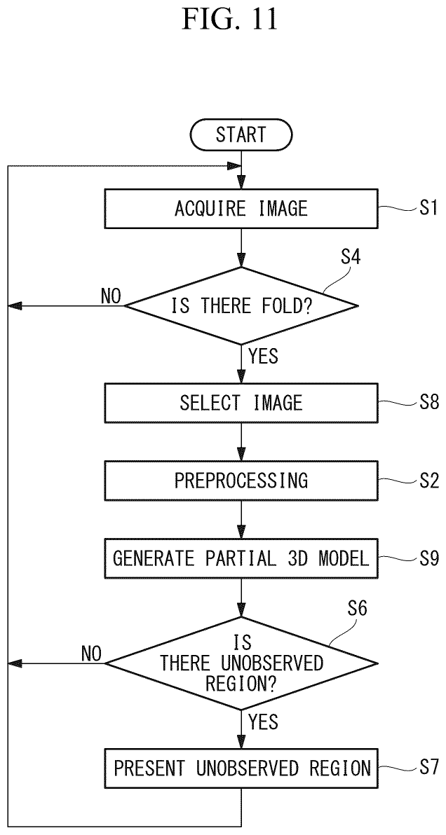

As shown in FIG. 11, the image processing method according to this embodiment includes steps S1, S2, S4, S6, S7, step S8 of selecting images in which the region of interest is detected, and step S9 of generating a partial 3D model from the selected images.

The image acquisition unit 11 sequentially acquires images input to the image processing apparatus 10 (step S1), and then the fold detection unit 14 detects the presence/absence of a fold B in each of the images acquired by the image acquisition unit 11 (step S4).

When no fold B is detected (NO in step S4), the process repeats steps S1 and S4 without performing steps S2 and S6 to S9.

Meanwhile, when a fold B is detected (YES in step S4), the image selection unit 18 selects images in which the fold B is detected, so that the images are used for 3D model generation. The preprocessing unit 12 preprocesses the selected images. The 3D reconstruction unit 13 generates a partial 3D model of the object A, which is a 3D model of the fold B and the peripheral portion thereof, from the plurality of preprocessed images (step S9).

Next, steps S6 and S7 are performed as in the first embodiment.

As described above, according to this embodiment, a partial 3D model is generated only from the images of the scenes including the region of interest. By using this partial 3D model for detection of an unobserved region, it is possible to prevent erroneous detection of an unobserved region and prevent erroneous information of an unobserved region from being presented to the user. In addition, it is possible to prevent information of an unobserved region from being excessively presented to the user.

Furthermore, because the region of interest is the region of a fold B, it is possible to effectively prevent the user from overlooking the reverse side of the fold B in the colonoscopy.

In addition, because the number of images used for generating a 3D model is limited, it is possible to reduce the amount of processing for generating the 3D model and to increase the processing speed, compared with the first embodiment.

### Third Embodiment

Next, an image processing apparatus, an image processing method, an image processing program, and a storage medium according to a third embodiment will be described.

This embodiment differs from the first embodiment in the method of generating a partial 3D model. In this embodiment, the configurations different from those in the first embodiment will be described, and the configurations common to those in the first embodiment will be denoted by the same reference signs, and the description thereof will be omitted.

As in the first embodiment, the image processing apparatus 10 according to this embodiment is applied to the endoscope system 100 including the endoscope 20, the control device 30, and the display device 40. The image processing apparatus 10 includes a processor 102, the storage unit 2, the memory 3, the input unit 4, and the output unit 5.

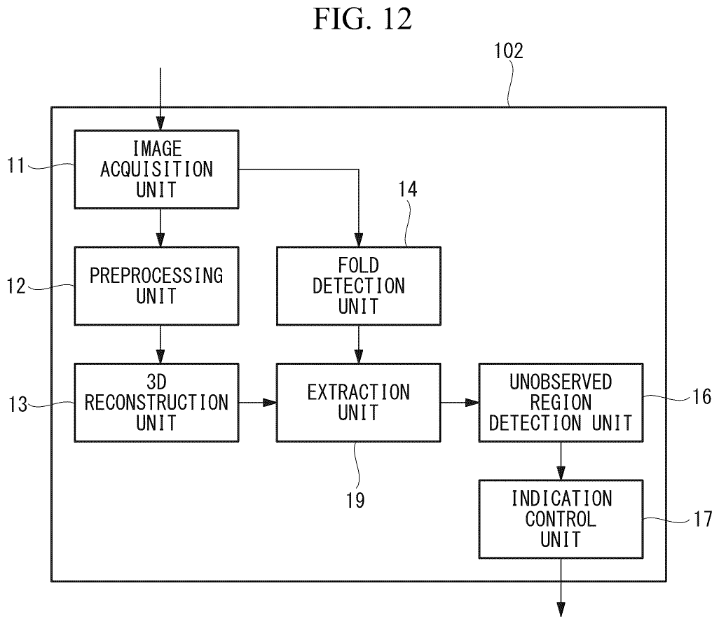

As shown in FIG. 12, the processor 102 includes, as functional units, an extraction unit 19 in addition to the preprocessing unit 12, the 3D reconstruction unit 13, the fold detection unit 14, the unobserved region detection unit 16, and the indication control unit 17.

The preprocessing unit 12, the 3D reconstruction unit 13, and the fold detection unit 14 perform processing using the images G acquired by the image acquisition unit 11, as in the first embodiment.

The extraction unit 19 extracts, from a whole 3D model D generated by the 3D reconstruction unit 13, a portion generated from the images in which a fold B is detected by the fold detection unit 14, to generate a partial three-dimensional model representing the fold B and the peripheral portion thereof.

The unobserved region detection unit 16 detects an unobserved region on the basis of the partial 3D model generated by the extraction unit 19.

Next, an image processing method performed by the image processing apparatus 10 will be described.

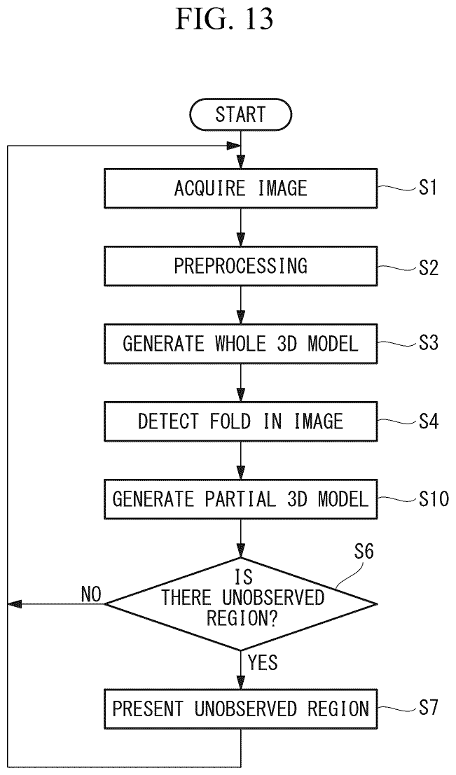

As shown in FIG. 13, the image processing method according to this embodiment includes steps S1 to S4, S6, S7, and step S10 of generating a partial three-dimensional model of the object A.

After steps S1 to S4 are performed as in the first embodiment, the extraction unit 19 extracts, from the whole 3D model D, a portion generated from the images in which a fold B is detected to obtain a partial 3D model of the object A (step S10).

Next, steps S6 and S7 are performed as in the first embodiment.

As described above, according to this embodiment, a partial 3D model formed only of the portion corresponding to the images of the scenes including the region of interest is generated. By using this partial 3D model for detection of an unobserved region, it is possible to prevent erroneous detection of an unobserved region and prevent erroneous information of an unobserved region from being presented to the user. In addition, it is possible to prevent information of an unobserved region from being excessively presented to the user.

Furthermore, because the region of interest is the region of a fold B, it is possible to effectively prevent the user from overlooking the reverse side of the fold B in the colonoscopy.

Furthermore, according to this embodiment, the partial 3D model includes the region of interest and the peripheral portion thereof. This improves the detection accuracy of an unobserved region in the region of interest and the peripheral portion thereof.

In each of the embodiments described above, the indication control unit 17 may change the indication H after an unobserved region C has been captured by the endoscope 20. After recognizing the presence of an unobserved region C on the basis of the indication H, the user moves the field of view F of the endoscope 20 and observes the unobserved region C. The processor 1, 101, 102 calculates, for example, the positional relationship between the position of the field of view F and the unobserved region C in the whole or partial 3D model D, and determines that the unobserved region C is captured if the unobserved region C is positioned in the field of view F.

After the unobserved region C is captured, the indication control unit 17 may erase the indication H (see FIGS. 5A to 6B), which may be an arrow, a marker, a frame, or a text. Alternatively, the indication control unit 17 may change the mode of the indication H. For example, the color of the frame may be changed (see FIG. 6A), or the text may be changed to “observation completed” or the like (see FIG. 6B).

When the unobserved region C that has been captured appears again in the image G, the indication control unit 17 may not indicate the indication H. Alternatively, the indication control unit 17 may indicate an indication showing that the unobserved region C has already been observed. For example, an arrow, a marker, or a frame (see FIGS. 5A to 6B) may be indicated in another color, or a text such as “already observed” may be indicated.

In each of the embodiments described above, the processor 1, 101, 102 does not necessarily have to perform the processing such as preprocessing, 3D reconstruction, and detection of a fold B on the entirety of each image G, and may perform the processing only on a portion selected from each image G.

In each of the embodiments described above, the predetermined region of interest is a fold B of the colon. However, the region of interest is not limited thereto, and may be any region in which the presence/absence of an unobserved region may need to be detected. In order to prevent a blind spot from being overlooked, the region of interest can be a region in which a blind spot in the field of view F of the endoscope 20 is likely to occur, and may be, for example, a tissue protruding from the inner surface of the lumen, such as a polyp.

Although embodiments and modifications have been described in detail with reference to the drawings, the specific configuration of the present disclosure is not limited to the embodiments and modifications described above, and various design changes can be made without departing from the scope of the present disclosure. The components mentioned in the embodiments and modifications described above may be combined as appropriate.

For example, the object may be a lumen other than the colon, or may be an organ other than the lumen that can be subjected to an endoscopic examination. The region of interest may be set depending on the object.

### REFERENCE SIGNS LIST

1 Processor
2 Storage unit (storage medium)
2a Image processing program
10 Image processing apparatus
20 Endoscope
A Object
C Unobserved region
D Whole three-dimensional model
E Missing part
F Field of view
G Image

What is claimed is:

1. An image processing apparatus comprising:
one or more processors comprising hardware, the one or more processors configured to:
acquire a plurality of images of an object captured by an endoscope;
detect a first region of the object included in the plurality of images;
generate a partial three-dimensional model of the object based on one or more images of the plurality of images, the partial three-dimensional model representing the first region; and
detect, based on the partial three-dimensional model, an unobserved region of the object that is not captured by the endoscope.

2. The image processing apparatus according to claim 1, wherein the first region is a tissue protruding from an inner surface of a lumen.

3. The image processing apparatus according to claim 1, wherein
the one or more processors are further configured to generate, based on the plurality of images, a whole three-dimensional model of the object included in the plurality of images,
the detecting of the first region includes detecting the first region from at least one of the whole three-dimensional model and each of the plurality of images, and
the generating of the partial three-dimensional model includes extracting a portion corresponding to the first region from the whole three-dimensional model.

4. The image processing apparatus according to claim 1, wherein
the detecting of the first region includes detecting the first region in each of the plurality of images, and
the generating of the partial three-dimensional model includes generating the partial three-dimensional model only from, among the plurality of images, images in which the first region is detected.

5. The image processing apparatus according to claim 1, wherein
the one or more processors are further configured to generate, based on the plurality of images, a whole three-dimensional model of the object included in the plurality of images,
the detecting of the first region includes detecting the first region in each of the plurality of images, and
the generating of the partial three-dimensional model includes extracting, from the whole three-dimensional model, a portion generated from the images in which the first region is detected.

6. The image processing apparatus according to claim 3, wherein
the detecting of the unobserved region includes detecting the unobserved region based on a positional relationship between the first region and a position of a field of view of the endoscope in the partial three-dimensional model or in the whole three-dimensional model of the object.

7. The image processing apparatus according to claim 1, wherein the detecting of the unobserved region includes detecting a missing part, as the unobserved region, in the partial three-dimensional model.

8. The image processing apparatus according to claim 1, wherein
the one or more processors are further configured to cause, when the unobserved region is detected, a display device to display in real time an indication indicating that the unobserved region is detected.

9. The image processing apparatus according to claim 8, wherein the indication is an arrow superimposed on the image for indicating a position of the first region in the plurality of images.

10. The image processing apparatus according to claim 8, wherein the indication is a marker superimposed at a position corresponding to the unobserved region in the plurality of images.

11. An image processing method comprising:
acquiring a plurality of images of an object captured by an endoscope;
detecting a first region of the object included in the plurality of images;
generating a partial three-dimensional model of the object based on one or more images of the plurality of images, the partial three-dimensional model representing the first region; and
detecting, based on the partial three-dimensional model, an unobserved region of the object that is not captured by the endoscope.

12. The image processing method according to claim 11, further comprises:
generating, based on the plurality of images, a whole three-dimensional model of the object included in the plurality of images,
wherein:
the detecting of the first region of interest includes detecting the first region of interest from at least one of the whole three-dimensional model and each of the plurality of images, and
the generating of the partial three-dimensional model includes extracting a portion corresponding to the first region from the whole three-dimensional model.

13. The image processing method according to claim 11, wherein
the detecting of the first region of interest includes detecting the first region in each of the plurality of images, and
the generating of the partial three-dimensional model includes generating the partial three-dimensional model only from, among the plurality of images, images in which the first region is detected.

14. The image processing method according to claim 11, further comprises:
generate, based on the plurality of images, a whole three-dimensional model of the object included in the plurality of images,
wherein:
the detecting of the first region includes detecting the first region in each of the plurality of images, and
the generating of the partial three-dimensional model includes extracting, from the whole three-dimensional model, a portion generated from the images in which the first region is detected.

15. The image processing method according to claim 12, wherein
the detecting of the unobserved region includes detecting the unobserved region based on a positional relationship between the first region and a position of a field of view of the endoscope in the partial three-dimensional model or in the whole three-dimensional model of the object.

16. A non-transitory computer-readable storage medium storing an image processing program, wherein the image processing program causes a computer to execute:
acquiring a plurality of images of an object captured by an endoscope;
detecting a first region of the object included in the plurality of images;
generating a partial three-dimensional model of the object based on one or more images of the plurality of images, the partial three-dimensional model representing the first region; and
detecting, based on the partial three-dimensional model, an unobserved region of the object that is not captured by the endoscope.

17. The non-transitory computer-readable storage medium according to claim 16, wherein the image processing program causes the computer to execute:
generate, based on the plurality of images, a whole three-dimensional model of the object included in the plurality of images,
wherein:
the detecting of the first region includes detecting the first region from at least one of the whole three-dimensional model and each of the plurality of images, and
the generating of the partial three-dimensional model includes extracting a portion corresponding to the first region from the whole three-dimensional model.

18. The non-transitory computer-readable storage medium according to claim 16, wherein
the detecting of the first region includes detecting the first region in each of the plurality of images, and
the generating of the partial three-dimensional model includes generating the partial three-dimensional model only from, among the plurality of images, images in which the first region is detected.

19. The non-transitory computer-readable storage medium according to claim 16, wherein the image processing programs causes the computer to execute
generating, based on the plurality of images, a whole three-dimensional model of the object included in the plurality of images, and
the detecting of the first region includes detecting the first region in each of the plurality of images, and
the generating of the partial three-dimensional model includes extracting, from the whole three-dimensional model, a portion generated from the plurality of images in which the first region is detected.

20. The non-transitory computer-readable storage medium according to claim 17, wherein
the detecting of the unobserved region includes detecting the unobserved region based on a positional relationship between the first region and a position of a field of view of the endoscope in the partial three-dimensional model or in the whole three-dimensional model of the object.
